(19) United States
(12) Patent Application Publication
(10) Pub. No.: US 2025/0006305 A1
(43) Pub. Date: Jan. 2, 2025

(54) METHODS AND SYSTEMS FOR AUTOMATED FRACTION SELECTION IN NUCLEIC ACID MANUFACTURING

(71) Applicant: Synthego Corporation, Redwood City, CA (US)

(72) Inventor: Puzhou Wang, Redwood City, CA (US)

(21) Appl. No.: 18/743,558

(22) Filed: Jun. 14, 2024

Related U.S. Application Data
(60) Provisional application No. 63/524,022, filed on Jun. 29, 2023.

(51) Int. Cl. G16B 30/10; G16B 40/10

(52) U.S. Cl. CPC G16B 30/10; G16B 40/10

(57) ABSTRACT

A method for manufacturing nucleic acid molecules, including: obtaining, using a processor, a plurality of fractions from a nucleic acid synthesis procedure; obtaining, using the processor, characterization information regarding each of the plurality of fractions, the characterization information including mass spectrometry and liquid chromatography data for each of the plurality of fractions; identifying, using the processor, a subset of the plurality of fractions to combine to generate a simulated pool based on a metric; simulating, using the processor, a predicted metric for the simulated pool based on identifying the subset of the plurality of fractions to combine; and providing, using the processor, information identifying the subset of fractions to a user to combine into a combined pool based on simulating the predicted metric for the simulated pool.

### CROSS-REFERENCE TO RELATED APPLICATIONS

This patent application claims the benefit of priority of U.S. Provisional Application No. 63/524,022, filed on Jun. 29, 2023, which is incorporated herein by reference in its entirety.

### BACKGROUND

In the manufacturing of nucleic acid molecules, such as guide RNA (gRNA) molecules, especially in the large-scale manufacturing, there are common in-line chromatography purification processes that will separate one crude input sample of the oligonucleotides into multiple fractions depending on the interaction strength between chemical species in the sample and chromatography columns used. Each of the resulting fractions will contain various chemical species at different concentrations. To move forward in the production workflow, some of the generated fractions will be combined based on specific constraints on parameters such as purity and yield. Such combination, also known as pooling, of fractions is often irreversible, which emphasizes the critical role of this fraction selection process.

To facilitate such fraction selection process, characterization methods such as mass spectrometry (MS) and liquid chromatography (LC) are commonly used to examine the quality of nucleic acid molecules, such as a gRNA molecule, in each fraction. Given chemistry knowledge of gRNA synthesis, algorithms have also been developed to quantify gRNA quality into MS and LC metrics for various fractions. Typically the data are analyzed manually to select which fractions to combine for moving forward. However, with an increasing number of fractions, manual examination of all characterization data, including both the traces and all metrics, quickly turns into a mundane or even impossible task. Consistent quality and yield suffer as a result.

Therefore, there is a need for a better fraction selection process to speed up the overall workflow without compromising the quality of the product during the process as well as the final product.

### SUMMARY

According to various embodiments, the present disclosure provides apparatus, systems, and methods for manufacturing nucleic acid molecules. One embodiment of the method includes obtaining information identifying a plurality of fractions from a nucleic acid synthesis procedure; obtaining characterization information regarding each of the plurality of fractions, where the characterization information may include mass spectrometry and/or liquid chromatography data for each of the plurality of fractions; identifying a subset of the plurality of fractions to combine to generate a simulated pool based on a metric; simulating a predicted metric for the simulated pool based on identifying the subset of the plurality of fractions to combine; and providing information identifying the subset of fractions to a user to combine into a combined pool based on simulating the predicted metric for the simulated pool.

In various embodiments, the apparatus, systems, and methods may be implemented using a processor (e.g., a microprocessor) which may be part of a local or networked computing system and which may be coupled to a non-transient computer-readable medium containing instructions for carrying out the procedures.

In some embodiments of the method the liquid chromatography data may include chromatogram data for each of the plurality of fractions, and simulating a predicted metric for the simulated pool may further include aligning the chromatogram data for each of the plurality of fractions, and aggregating the aligned chromatogram data for each of the plurality of fractions based on aligning the chromatogram data to produce simulated pool chromatogram data. In certain embodiments of the method, simulating a predicted metric for the simulated pool may further include identifying a plurality of peaks in the simulated pool chromatogram data, determining a main peak in the simulated pool chromatogram data based on identifying the plurality of peaks, and determining the predicted metric for the simulated pool based on determining the main peak.

In particular embodiments of the method, obtaining characterization information may further include obtaining characterization information including mass spectrometry data for each of the plurality of fractions. In various embodiments of the method, simulating a predicted metric for the simulated pool may further include simulating the predicted metric for the simulated pool based on determining a weighted average of the mass spectrometry data for the subset of the plurality of fractions. In some embodiments of the method, determining a weighted average of the mass spectrometry data of the subset of the plurality of fractions may further include weighting the average of the mass spectrometry data for the subset of the plurality of fractions based on a molarity of nucleic acids in each fraction of the subset of the plurality of fractions.

In some embodiments of the method, simulating a predicted metric for the simulated pool may further include generating a predicted mass spectrometry spectrum for the simulated pool. In particular embodiments of the method, simulating a predicted metric for the simulated pool may further include simulating a plurality of predicted metrics for the simulated pool, and generating a table of the plurality of predicted metrics.

In various embodiments of the method, identifying a subset of the plurality of fractions to combine to generate a simulated pool may further include adding a fraction to the subset of fractions to generate a new subset of fractions based on determining the weighted average of the metric of the subset of the plurality of fractions, and determining an updated metric for the new subset of the plurality of fractions.

In some embodiments of the method, providing information identifying the subset of fractions to a user to combine into a combined pool may further include receiving input from the user selecting a modified subset of fractions to combine into the combined pool, where the modified subset of fractions may be different from the subset of fractions.

In particular embodiments of the method, identifying a subset of the plurality of fractions to combine to generate a simulated pool based on a metric may further include identifying a subset of the plurality of fractions to combine to generate a simulated pool based on identifying the simulated pool having a local optimum value.

Various embodiments of the method may further include combining the subset of fractions into the combined pool. Some embodiments of the method may further include further processing the combined pool to generate a nucleic acid product. In certain embodiments of the method, the nucleic acids and/or nucleic acid product may include guide RNA (gRNA) molecules.

Some embodiments of the method may further include combining a portion of each of the subset of fractions into a mock combined pool; obtaining further characterization information from the mock combined pool, where the further characterization information may include at least one of liquid chromatography data or mass spectrometry data for the combined pool; and determining, based on the further characterization information, whether the mock combined pool satisfies a quality metric for the nucleic acid molecules.

### BRIEF DESCRIPTION OF THE DRAWINGS

The detailed description is described with reference to the accompanying figures. The use of the same reference numbers in different instances in the description and the figures may indicate similar or identical items. Various embodiments or examples of the present disclosure are disclosed in the following detailed description and the accompanying drawings. The drawings are not necessarily to scale. In general, operations of disclosed processes may be performed in an arbitrary order, unless otherwise provided in the claims.

FIG. 1A provides an example of a workflow overview where the Fraction Analyzer is used for pooling of fractions to proceed in a manufacturing process.

FIG. 1B is a flowchart showing the workflow of fraction analysis with MS- and/or LC-based algorithms.

FIG. 2 is a block diagram schematically showing the configuration of the MS-based fraction selection algorithms with their data input according to the present invention.

FIG. 3A is a block diagram schematically showing the configuration of the LC-based fraction selection algorithms with their data input according to the present invention. In one configuration, the algorithm includes an optional step of providing a yield vs. purity threshold plot or graph (FIG. 3B).

FIG. 4 is a flowchart showing a process of generating the optimal pool based on the constraint according to the present invention.

FIG. 5 is a flowchart showing the workflow of pool chromatogram predictor.

FIG. 6 is a flowchart showing the workflow of auto integrator.

FIG. 7 shows the distribution of metric value differences in a validation dataset composed of unique sets of guide RNA (gRNA) fractions from two different batches.

FIG. 8 is a table showing a comparison of spec values between the real pool sample (designated as real) and the pool sample simulated by Fraction Analyzer (designated as sim).

FIG. 9A shows the full spectrum of real (top panel) and simulated (bottom panel) as well as a zoomed in version around desired full-length product (FIG. 9B).

FIG. 10 is a line plot showing one example comparison between observed and predicted pool chromatogram.

FIG. 11 is a scatter plot showing comparison of LC purity values reported by commercial software and auto integrator.

FIG. 12 is a histogram plot showing the differences of LC purity values reported by commercial software and auto integrator.

FIG. 13 shows an example of an editable table with pre-populated fraction number, an empty column of concentration, and a pre-populated column of volume.

FIG. 14 shows an example of a concentration csv file containing a column with the name conc for the storage of concentration values, a column with the name volume for the storage of volume values, a column of esi_well_label, and a column of fraction.

FIG. 15 shows an example of a table with checkboxes in front of each fraction; pre-selected fractions are the suggested pool decision that maximize pool yield and meet the MS metric spec limits, color-coded e.g., red (Fractions 1 to 9, and 29 to 44), orange (Fractions 25 to 28), and green (Fractions 10 to 24) to show the quality of each fraction.

FIG. 16 shows an example of a simulated spec table based on user's selection in the Fraction Selector.

FIG. 17 shows an example of a simulated pool spectrum based on user's selection in the Fraction Selector.

FIG. 18 shows the switch tab in the web-based application, Fraction Analyzer, where a user can choose between the MS- and LC-based algorithms.

FIG. 19 shows an example of an editable table with pre-populated columns of fraction number, estimated purity, concentration, and volume.

FIG. 20 shows an example of an overlay of LC chromatogram data for all the fractions involved in the LC-based selection process.

FIG. 21 shows an example of the interface in LC-based fraction selection algorithms for a user to define global integration parameters.

FIG. 22 shows an example of a table with checkboxes in front of each fraction; pre-selected fractions are the suggested pool decision that maximize pool yield and meet the LC metric spec limit.

FIG. 23 shows an example of a simulated pool chromatogram based on user's selection in the fraction selection table.

FIG. 24 shows an example of the interface in LC-based fraction selection algorithms for a user to define integration parameters used in the simulated pool chromatogram.

FIG. 25 shows an example of a yield vs. purity threshold plot or graph as may be generated in the optional step of FIG. 3B.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

As used herein a letter following a reference numeral is intended to reference an embodiment of the feature or element that may be similar, but not necessarily identical, to a previously described element or feature bearing the same reference numeral (e.g., 1, 1a, 1b). Such shorthand notations are used for purposes of convenience only and should not be construed to limit the disclosure in any way unless expressly stated to the contrary.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of “a” or “an” may be employed to describe elements and components of embodiments disclosed herein. This is done merely for convenience and “a” and “an” are intended to include “one” or “at least one,” and the singular also includes the plural unless it is obvious that it is meant otherwise.

Finally, as used herein any reference to “an embodiment”, “one embodiment” or “some embodiments” means that a particular element, feature, structure, or characteristic described in connection with the embodiment is included in at least one embodiment disclosed herein. The appearances of the phrase “in some embodiments” in various places in the specification are not necessarily all referring to the same embodiment, and embodiments may include one or more of the features expressly described or inherently present herein, or any combination of sub-combination of two or more such features, along with any other features which may not necessarily be expressly described or inherently present in the instant disclosure.

A “nucleic acid molecule” as used herein, can generally refer to a polymeric form of nucleotides of any length, either ribonucleotides and/or deoxyribonucleotides. Thus, these terms include, but are not limited to, single-, double-, or multi-stranded DNA or RNA, genomic DNA, complementary DNA (cDNA), guide RNA (gRNA), messenger RNA (mRNA), DNA-RNA hybrids, or a polymer including purine and pyrimidine bases or other natural, chemically or biochemically modified, non-natural, or derivatized nucleotide bases.

The term “guide RNA” or “gRNA,” as used herein, generally refers to an RNA molecule (or a group of RNA molecules collectively) that can bind to a Cas protein and aid in targeting the Cas protein to a specific location within a target polynucleotide (e.g., a DNA). A guide RNA can include a CRISPR RNA (crRNA) segment and a trans-activating crRNA (tracrRNA) segment. The term “crRNA” or “crRNA segment,” as used herein, can refer to an RNA molecule or portion thereof that includes a polynucleotide-targeting guide sequence, a stem sequence, and, optionally, a 5′-overhang sequence. crRNA is described, for example, by Jiang et al. (Nat Biotechnol. 2013 March; 31 (3): 233-239) and Jinek et al. (2012, Science, 337:816-821). The term “tracrRNA” or “tracrRNA segment,” can refer to an RNA molecule or portion thereof that includes a protein-binding segment (e.g., the protein-binding segment is capable of interacting with a CRISPR-associated protein, such as a Cas9). The term “guide RNA” as used herein encompasses a single guide RNA (sgRNA), where the crRNA segment and the tracrRNA segment are located in the same RNA molecule as described, for example, by Jinek et al. (2012, Science, 337:816-821).

Described herein are automated fraction selection methods for determining which fractions of a nucleic acid molecule preparation can be optimally recombined during a manufacturing process.

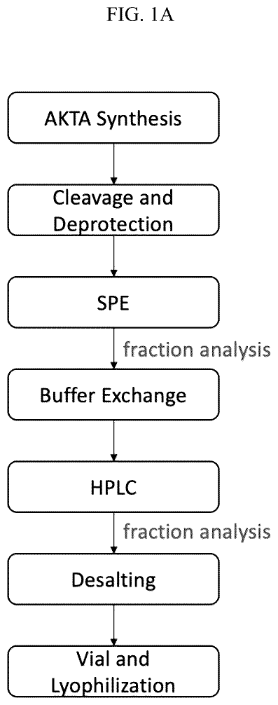

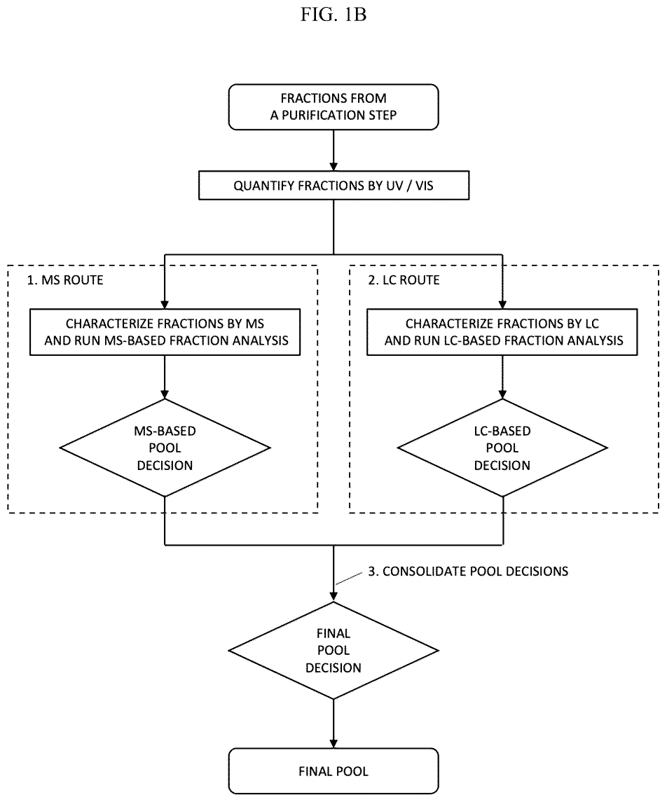

### FIG. 1A

FIG. 1A illustrates a manufacturing workflow for guide RNA manufacturing. gRNA is synthesized using an AKTA Synthesizer (Cytiva), for example, and undergoes purification to ensure a desired quality. After cleavage and deprotection the gRNA synthesis preparation undergoes solid-phase extraction (SPE), buffer exchange, high-performance liquid chromatography (HPLC) purification, desalting, and then vial filling and lyophilization. The fractions are generated during SPE and HPLC. Therefore, the Fraction Analyzer methods disclosed herein can be used after either or both of these steps for Fraction Selection (i.e., to pool fractions for the next step in the workflow), or other steps as appropriate for a particular process. It is to be understood that embodiments of the methods disclosed herein may include one or more of the steps described herein. Further, such steps may be carried out in any desired order and two or more of the steps may be carried out simultaneously with one another. Two or more of the steps disclosed herein may be combined in a single step, and in some embodiments, one or more of the steps may be carried out as two or more sub-steps. Further, other steps or sub-steps may be carried in addition to, or as substitutes to one or more of the steps disclosed herein.

In certain embodiments, methods disclosed herein include mass spectrometry (MS) and/or liquid chromatography (LC) based algorithms to examine the quality of nucleic acid molecules, such as a gRNA molecule, in each fraction, as illustrated, for example, in FIG. 1B.

### FIG. 1B

FIG. 1B provides a flowchart having: rectangular shapes with round corners used to represent physical RNA samples such as a plurality of fractions and a pool composed by the subset of the plurality of fractions; rectangular shapes with square corners are used to represent procedures; diamond shapes are used to represent pool decisions, without the physical combination of the identified fractions. UV/Vis stands for ultraviolet-visible spectroscopy, which is a technology widely used in the field of nucleic acid manufacturing for quantification of molarity. MS stands for mass spectrometry. LC stands for liquid chromatography. Even though MS and LC routes are shown as being used complementarily in this flowchart, they can in fact be run independently (e.g., in the use case of fraction analysis based on LC data only) or sequentially (e.g., in the use case of first filtering fractions by MS route and then only pass the filtered fractions through LC route).

### 1. MS Route

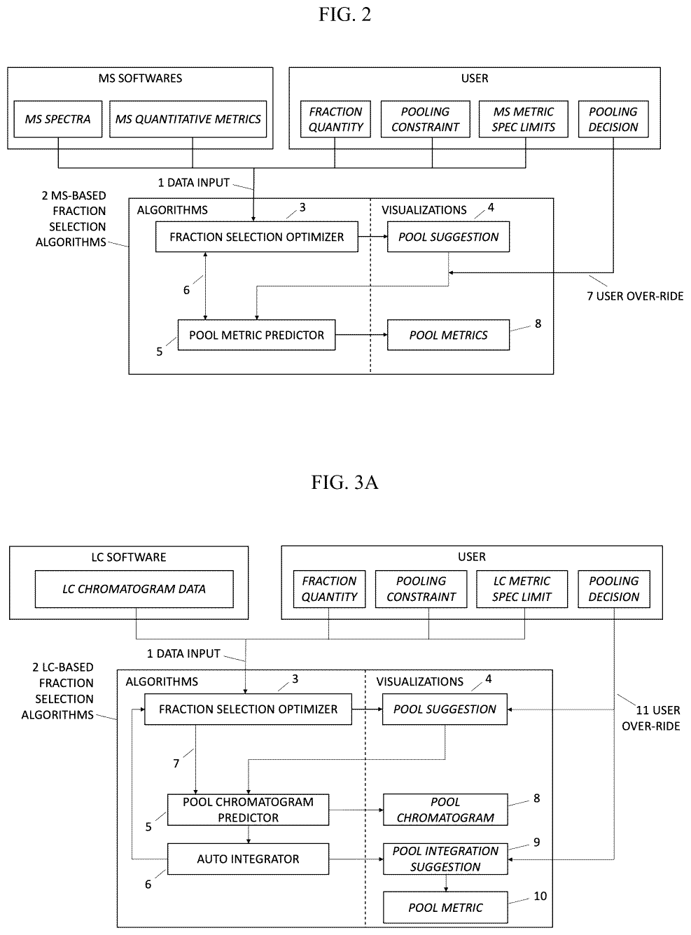

This the method involves the MS-based algorithms described in FIG. 2.

### 2. LC Route

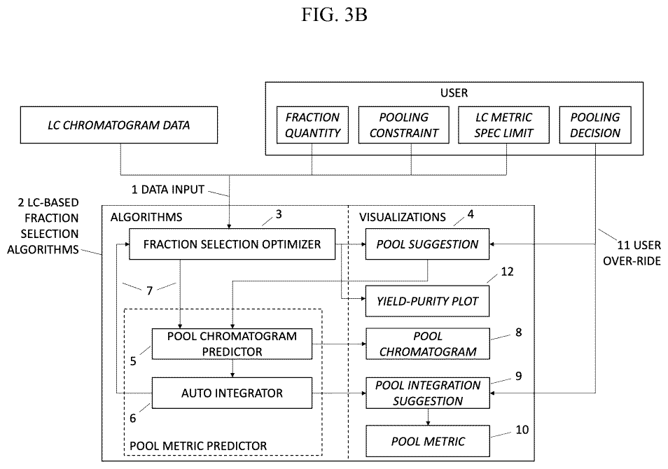

Characterization information, for example chromatogram data, is first obtained regarding each of the plurality of fractions. Such characterization information is then used in the LC-based fraction analysis algorithms to provide user with pool suggestions. Detailed workflow is described in FIGS. 3A and 3B.

### 3. Consolidation of Pool Decisions

When both MS and LC routes are used in the fraction analysis process, there will be a consolidation of the pool decisions from both routes to determine the final pool decision. A common, but not exclusive, logic is the intersection of the two subsets of fractions decided by the two routes. E.g., if MS-based and LC-based pool decisions are {2,3,4,5} and {4,5,6}, respectively, the final pool decision will be {4,5}.

### FIG. 2

FIG. 2 is a block diagram schematically showing the configuration of the MS-based fraction selection algorithms with their data input according to the present invention.

Each box illustrated in FIG. 2 represents one module in the workflow of MS-based fraction selection algorithms. Italic names refer to input and output data, while regular names refer to module and submodule names. At step 1 Data Input, the input data to MS-based fraction selection algorithms include, MS spectra data and MS quantitative metrics obtained from MS quantification software such as the commercially available software MassHunter (Agilent Technologies, Inc.); fraction quantity provided by user; pooling constraint provided by user, e.g., maximization of pool yield; and MS metric spec limits, used to determine the desired range for each MS quantitative metrics.

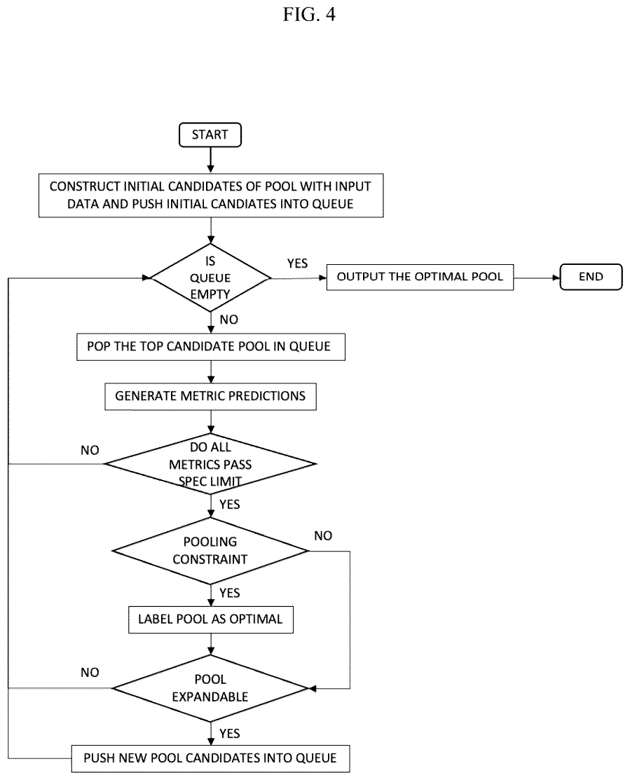

MS-based fraction selection algorithms 2 (FIG. 2) used in methods disclosed herein are split into two groups: algorithms and visualizations. The former includes fraction selection optimizer based on pooling constraint mentioned in 1, as well as pool metric predictor, which are shown in FIG. 4 and Equation 1, respectively. The latter group includes a visualization of pool suggestion, where user can over-ride with manual input of preferred pool decision, and a visualization of the predicted metrics based on the current pool decision.

The Fraction Selection Optimizer algorithm 3 (FIG. 2) optimizes pool decisions based on one or more constraint from a user. This algorithm is further described in FIG. 4.

A Pool Suggestion is provided from the Fraction Selection Optimizer 4 (FIG. 2). This is the output from fraction selection optimizer 3 (FIG. 2), which is visualized as checked boxes in a selectable Dash DataTable described herein.

The Pool Metrics Predictor 5 (FIG. 2) is an algorithm to predict MS metrics for any pool decision. For more details, please refer to Equation 1 below.

As part of the algorithm, fraction selection optimizer 3 (FIG. 2) will pass information 6 (FIG. 2) to pool metric predictor 5 (FIG. 2), which will feed predictions back to the optimizer algorithm 3 (FIG. 2) as shown also in FIG. 4.

Upon review of the pool suggestion provided by fraction selection optimizer 3 (FIG. 2), user can decide to over-ride such suggestion by check and uncheck certain fractions listed in the Dash Data Table used to visualize the pool suggestion 7 (FIG. 2).

A tabulated output of pool metric predictor with fill colors coded based on the MS metric spec limits provided by user provides the final Pool Metrics 8 (FIG. 2). Colors can be assigned as desired by the user, for example, transparent can indicate that the predicted metric passes its spec limit; orange can indicate that the predicted metric passes but within 20% margin; red can indicate that the predicted metric fails its spec limit.

To achieve an algorithmic workflow of fraction selection with MS data, there are two major problems that need to be solved: (A) the reliable prediction of MS data for combined fractions (i.e., pools); and (B) the optimization of fraction selections with specific constraints. Based on the understanding of gRNA MS, to solve the prediction problem (i.e., problem A) a weighted average algorithm was developed (Equation 1), where the weight was the molarity of gRNA in each fraction.

### Equation 1:

$${metric}_{P} = \frac{{\sum }_{f ∈ P} ( {n}_{f} \times {metric}_{f} )}{{\sum }_{f ∈ P} {metric}_{f}} . \qquad ( 1 )$$

In the above equation, P represents a specific pool as a set of individual fractions, {f₁, f₂, f₃, . . . fₖ}; n_{f} represents the molarity of gRNA in one fraction f, metricₚ and metrics represent one MS metric for a pool P and one fraction f, respectively.

For the constrained optimization of fraction selections, i.e., problem B mentioned in the previous paragraph, it was converted to a multidimensional knapsack problem (MKP). It is well known that MKP is an NP-hard problem. Thus, given N fractions and each fraction with M unique MS metrics, the time complexity to optimize fraction selections with constraints is O(M×2^{N}). In real-life manufacturing, it is not uncommon to track dozens of MS metrics for dozens of fractions simultaneously, which leave the constrained optimization of fraction selections impractical. To mitigate such issue, the local optimum for MKP in fraction selection optimization was solved, which is consistent with the chemical understanding of gRNA chromatography. The time complexity of local optimal solution with N fractions and M unique MS metrics can be dramatically reduced to O(M×N²), which is far more practical with modern computational systems.

### FIGS. 3A and 3B

FIGS. 3A and 3B are flowcharts where each box represents one module in the workflow of LC-based fraction selection algorithms. Italic names refer to input and output data, while regular names refer to module and submodule names.

### 1. Data Input

In this step, the input data to LC-based fraction selection algorithms include one or more of:
LC chromatogram data obtained from a commercially available LC software such as Agilent OpenLab ChemStation or Cytiva UNICORN;
fraction quantity quantified by UV/Vis and provided by user;
pooling constraint(s) provided by user, e.g., maximization of pool yield; and/or
LC metric spec limit, usually a lower threshold for LC purity value.

### 2. LC-Based Fraction Selection Algorithms

The algorithms are split into two groups: algorithms and visualizations. The most important difference between LC-based algorithms and MS-based algorithms (FIG. 2) is that the function of pool metric prediction is now performed by two algorithms, pool chromatogram predictor (5) and auto integrator (6), instead of a single component as in the MS-based algorithms. Such difference is caused by the inherent differences in LC and MS data (i.e., LC data is time-based and MS data is based on molecular weight).

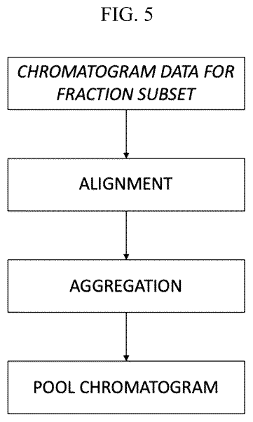

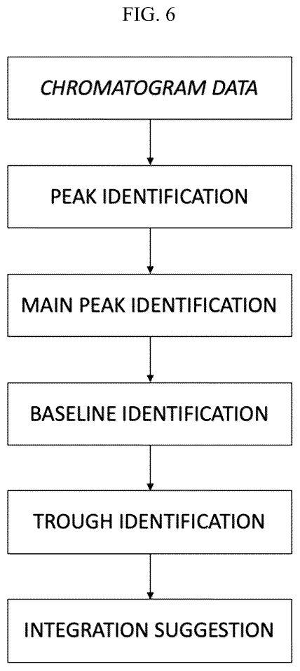

The algorithms include:
fraction selection optimizer (3) based on pooling constraint mentioned in “1. Data input” above (see FIG. 4), and
pool metric predictor module, including one or both of:
   pool chromatogram predictor (5), explained in FIG. 5, and/or
   auto integrator (6), explained in FIG. 6.

The visualizations include one or more of:
visualization of pool suggestion (4), where user can over-ride with manual input of a preferred pool decision,
visualization of the simulated pool chromatogram (8),
suggestion of integration parameters for the pool chromatogram (9),
visualization of pool metric (10), i.e., LC purity value for the simulated pool chromatogram, and/or
visualization of optimal pool yields at different LC metric spec limits (12).

### 3. Fraction Selection Optimizer

This is the core algorithm to optimize pool decisions based on constraint from user as described in FIG. 4.

### 4. Pool Suggestion

This is the output from fraction selection optimizer (3), which is visualized as checked boxes in a selectable Dash DataTable.

### 5. Pool Chromatogram Predictor

This is the core algorithm to predict the pool chromatogram for any pool decision. For more details, please refer to FIG. 5.

### 6. Auto Integrator

This is the core algorithm to suggest integration parameters based on a chromatogram. The suggested parameters will be then used to calculate LC purity (Eq. 2):

$$LC purity = \frac{{\sum }_{t ∈ [ {t}_{main start} , {t}_{main end} ]} ℱ ( t )}{{\sum }_{t ∈ [ {t}_{total start} , {t}_{total end} ]} ℱ ( t )} \times 100 \qquad ( 2 )$$

Eq. 2 is an equation showing the calculation of LC metric, i.e., LC purity of a chromatogram.

### 7. Dependence of Fraction Selection Optimizer (3) on Pool Chromatogram Predictor (5) and Auto Integrator (6)

As part of the algorithm, fraction selection optimizer (3) will pass information to pool chromatogram predictor (5), which will then provide a predicted chromatogram to auto integrator (6). Auto integrator (6) will then feed prediction of pool metric back to the optimizer algorithm (3). Pool chromatogram predictor (5) and auto integrator (6) together serve as a metric predictor module.

### 8. Pool Chromatogram

This is the output from pool chromatogram predictor (3), which is visualized as an interactive Dash Graph.

### 9. Pool Integration Suggestion

This is the output from auto integrator (6), which is a combination of one or more of: t_(total start), t_(total end), t_(main start), and/or t_(main end) (see Eq. 2).

### 10. Pool Metric

This is the calculated LC purity of pool chromatogram (see Eq. 2).

### 11. User Over-Ride

In LC-based algorithms, user can over-ride pool suggestion (4) and/or pool integration suggestion (9). To over-ride the pool suggestion provided by fraction selection optimizer (3), user can check and/or uncheck certain fractions listed in the Dash DataTable used to visualize the pool suggestion. To over-ride the pool integration suggestion (9), user can simply type in new values.

### 12. Yield-Purity Plot

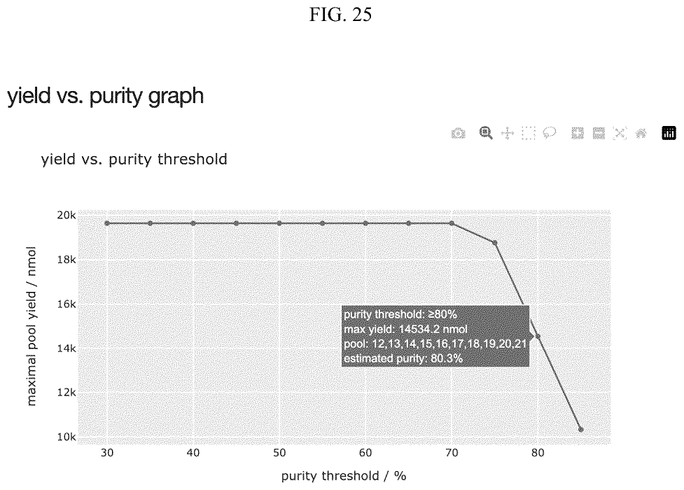

As shown in FIG. 3B, this optional configuration provides the output from fraction selection optimizer (3), which is visualized as an interactive Dash Graph (e.g., see FIG. 25).

### FIG. 4

FIG. 4 provides a flowchart summarizing implementation of a method disclosed herein. Characterization data are used to select the fractions to form initial pool candidates. The fractions are selected according to the preselected metric specification criteria. Some examples of metric specification criteria include that the percentage of n−1 peaks cannot exceed 10%, and that the percentage of desired product peak must be over 50%; however, other metric specification criteria are possible. The individual selected fractions will form the initial pool candidates. E.g., within a set of fractions numbered as #1, #2, #3, and #4, if fractions #2 and #3 pass metric specification criteria, they will form two initial pool candidates, namely, {#2} and {#3}. If the queue is empty the output is the optimal pool, which will be visualized as the Pool Suggestions for the user to review. If the queue is not empty, the candidate pool at the top of the queue is removed from the queue and examined, metric predictions are generated and analyzed by the software according to preselected metric specification criteria. If the metrics do not pass the specification limit, the flow is started over again with the next candidate pool at the front of the queue. If the pool meets the desired specification limits, the flow moves to compare its performance with the current optimal candidate within the Pooling Constraint, e.g., the current pool with the highest yield or other preselected metric(s). If out-performing current optimal pool regarding the Pool Constraint, the candidate pool will be labeled as the new optimal. If not, the current optimal pool stays the same. The software will then determine if the pool is expandable. If Yes, then the pool is expanded, and its expanded versions will form new candidate pools that are pushed back into the queue. If not expandable, the process is restarted with the next top candidate.

As used herein, a pool is “expanded” by including into the pool the next adjacent fraction based on fraction numbering. E.g., within a set of fractions numbered as #1, #2, #3, and #4, a pool candidate of {#2, #3} will be deemed as expandable on both lower and higher ends to form new candidate pools, {#1, #2, #3} and {#2, #3, #4}, respectively. Within that same case, a pool candidate of {#1, #2} will be only expandable on the higher end to form a new candidate, {#1, #2, #3}, while a candidate of {#3, #4} will be only expandable on the lower end. The candidate, {#1, #2, #3, #4}, will be not expandable.

For testing the algorithms and methods disclosed herein, a Fraction Analyzer tool was built upon a public python library called Dash. This tool was developed to help large-scale production of guide RNA molecules, especially to streamline the process of fraction quality evaluation and pool decision making.

As described herein, Fraction Analyzer starts with user input of a work order ID that represents the MS and/or LC run of a specific batch of fractions. After a quick validation of the work order ID, Fraction Analyzer generates an editable table of all the fractions found in the process run and allows user input for further information such as oligo concentration in each fraction. After the concentration information is gathered, the algorithm automatically calculates the maximum number of fractions for pooling, with the criteria being that the pool sample should pass all the metric specs. Finally, Fraction Analyzer provides user with a selectable table of fractions, as well as simulated metric values and simulated spectrum/chromatogram for the pool sample. If the fraction selection is overridden by a user, Fraction Analyzer can update the simulated metric values and spectrum/chromatogram accordingly.

### FIG. 5

FIG. 5 is a flowchart showing the workflow of pool chromatogram predictor. The input for pool chromatogram predictor is the chromatogram data for each individual fraction in a plurality of fractions. The first step in the predictor is alignment of all the chromatogram data. Alignment is especially important if the raw chromatogram data are irregularly sampled and need to be aligned to a common time grid or resampled at regular intervals. Alignment algorithms applicable to LC chromatogram data include, but are not limited to, linear interpolation, spline interpolation, and Gaussian process regression. The next step in pool chromatogram predictor is aggregation by the aligned time grid. Aggregation methods can range from simple aggregation such as sum or mean, to model-based aggregation to capture a priori chemical knowledge involved in the chromatogram data. The aggregation result will be used as the output of the predictor, i.e., the pool chromatogram.

### FIG. 6

FIG. 6 is a flowchart showing the workflow of auto integrator. The auto integrator algorithm is used to estimate LC metric, i.e., LC purity, for both the simulated pool chromatogram data and the chromatogram data of each fraction. The input for auto integrator is the data for one chromatogram, which can be represented as a time series, [symbol] (t), where t∈[Tₛₜₐᵣₜ, Tₛₜₐᵣₜ+Δt, . . . , T_{end}]. Tₛₜₐᵣₜ and T_{end} are the start and end time stamps of the chromatogram, respectively, while Δt represents the sampling time interval. The first step in auto integrator is peak identification to find the timestamps of all the N peaks in [symbol] (t), tₚₑₐₖ_{ᵢ}, where i=1, 2, . . . , N. The definition of a peak is that [symbol] (tₚₑₐₖ_{ᵢ})> [symbol] (tₚₑₐₖ_{ᵢ}−Δt) and [symbol] (tₚₑₐₖ_{ᵢ})> [symbol] (tₚₑₐₖ_{ᵢ}+Δt). Then the algorithm will identify the main peak, peak main, so that

$$ℱ ( {t}_{{peak}_{main}} ) = {max}_{i} ℱ ( {t}_{{peak}_{i}} ) .$$

After the identification of the main peak, the algorithm will then identify the baseline timestamps, t_{total start} and t_{total end}, by the definition

∀t∈[t_{total start},t_{total end}], [symbol] (t)≥δ· [symbol] (tₚₑₐₖₘₐᵢₙ)

where δ is a parameter defined (e.g., 0.01). The next step in auto integrator is to identify the trough points before and after tₚₑₐₖ_{ₘₐᵢₙ}, i.e., t_{main start} and t_{main end}, respectively. The definition of t_{main start} is that t_{main start} E [tₚₑₐₖ main-1′ tₚₑₐₖ main.] and [symbol] (t_{main start})< [symbol] (t_{main start}−Δt) and [symbol] (t_{main start})< [symbol] (t_{main start}+Δt). Similarly, the definition of t_{main end} is that t_{main end} ∈[tₚₑₐₖ_{ₘₐᵢₙ}′ tₚₑₐₖ _{ₘₐᵢₙ₊₁}] and [symbol] (t_{main end})< [symbol] (t_{main end}−Δt) and [symbol] (t_{main end})< [symbol] (t_{main end}+Δt). The set of parameters, t_{total start}, t_{total end}, t_{main start}, and t_{main end} will be the output of auto integrator and used for the calculation of pool metric, i.e., the LC purity of pool chromatogram (see Eq. 2).

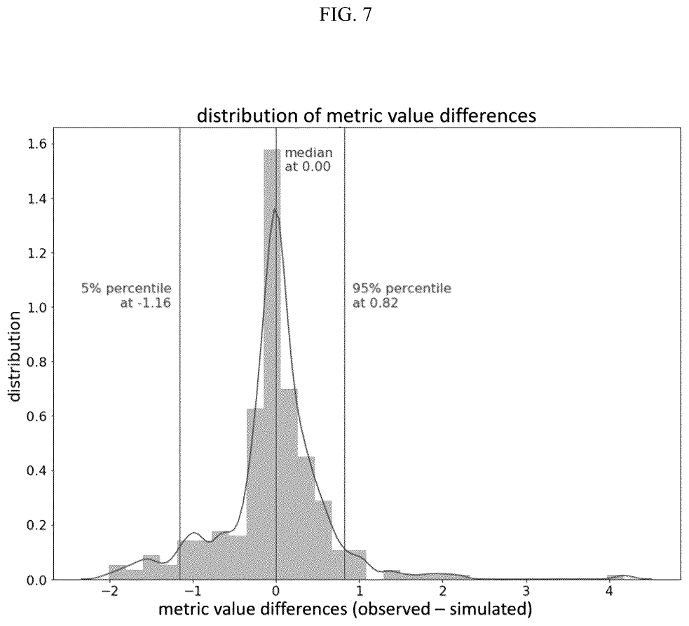

The weighted average algorithm disclosed herein was extensively tested with validation datasets. Illustrated in FIG. 7 is the distribution of metric value differences found in the validation dataset that is composed of 11 unique sets of gRNA fractions from two different batches. As demonstrated in the figure, 90% of the metric value differences were within +1%, with the mode of differences centered at 0%.

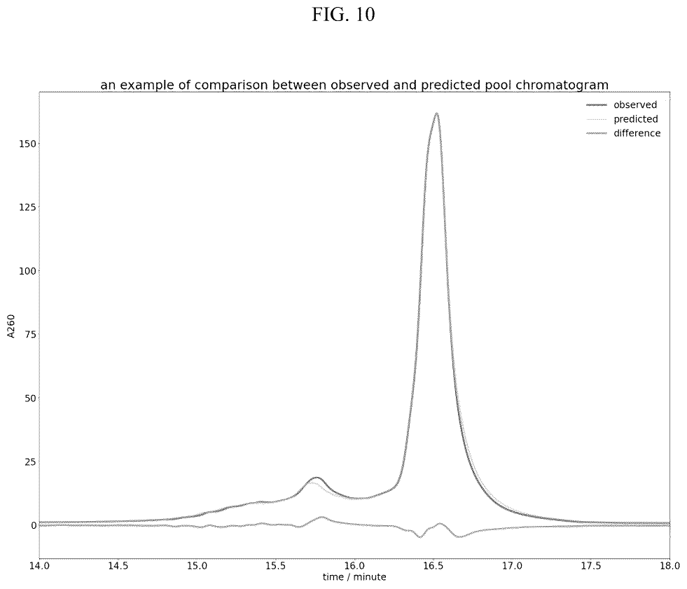

Three pool samples have been used to validate the pool chromatogram predictor algorithms, with r² values calculated between observed and predicted chromatograms as 0.998, 0.999, and 0.997. Shown in FIG. 10 is the comparison with the lowest r² value at 0.997. Both the r² value and the overlapping between observed and predicted traces showed the robustness of the predictor algorithms.

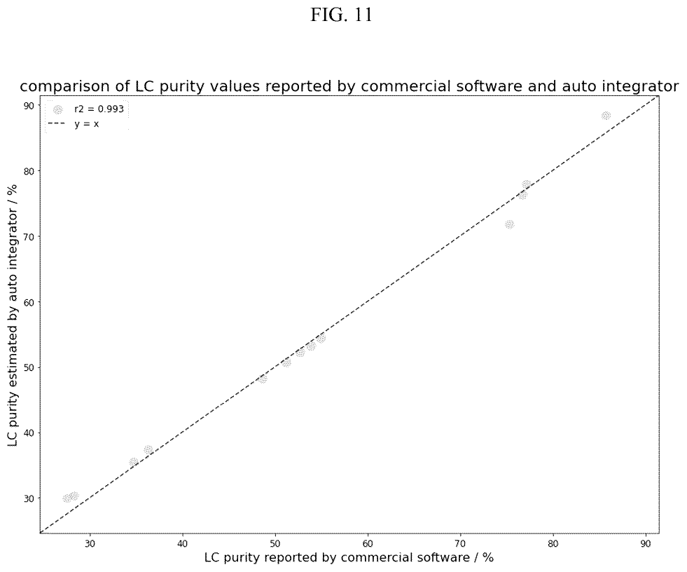

A total of 13 samples were used in the comparison, with LC purity values reported by commercial software ranging from 28% to 86%. As shown in the scatter plot in FIG. 11, the LC purity values reported by auto integrator were very close to those reported by commercial software, suggesting that the auto integrator algorithm proposed can be used reliably to estimate pool quality.

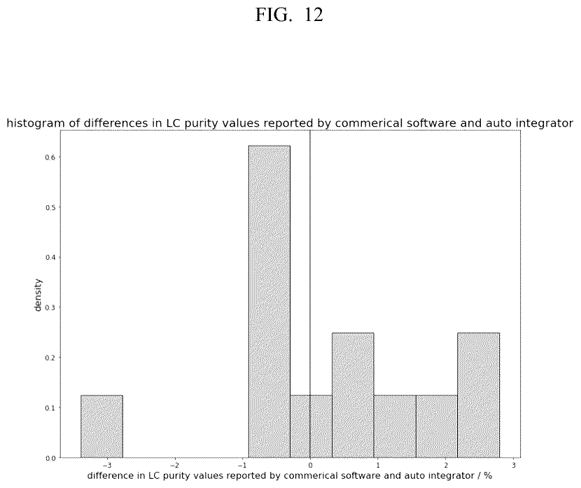

Shown in FIG. 12 are the plotted differences in LC purity values observed for the 13 samples plotted in FIG. 11. As seen in the histogram, all the difference fell between [−3.5%, 2.8%], re-emphasizing that the auto integrator algorithm proposed can be used reliably to estimate pool quality.

The reduction of time complexity in MKP was not intentionally validated, because the latency in response time of the web-based application was understood without such reduction.

The run-time for local optimum solution is usually 1-2 minutes, which led to an estimation of 4-8 hours for the run-time without the reduction in time complexity.

With the unique solutions provided to the problems of MS data prediction and fraction selection optimization, the algorithm-based method disclosed herein streamlined the fraction selection process based on mass spectrometry data and achieves reliable results.

### EXAMPLE

The following are a list of non-limiting examples of the disclosed procedures in accordance with one or more embodiments.

A batch of guide RNAs was synthesized using an AKTA oligopilot 100 (Cytiva). The batch was processed in cleavage step to detach crude RNA sample from the solid support used in synthesis. The crude RNA sample then underwent deprotection to remove any chemical protecting groups used during synthesis. The deprotected RNA sample was subjected to Solid Phase Extraction (SPE) and fractions were collected. The fractions underwent electrospray ionization (ESI) MS analysis and the data were collected for each fraction. MS-based fraction analysis on these SPE fractions was performed similarly to the example process described below, and thus omitted here. The subset of SPE fractions were then combined into a pool. The post-SPE pool sample underwent buffer exchange process to get ready for another purification step, high-performance liquid chromatography (HPLC). During the HPLC step, fractions were again collected. These HPLC fractions were characterized by MS and the data were collected for each fraction.

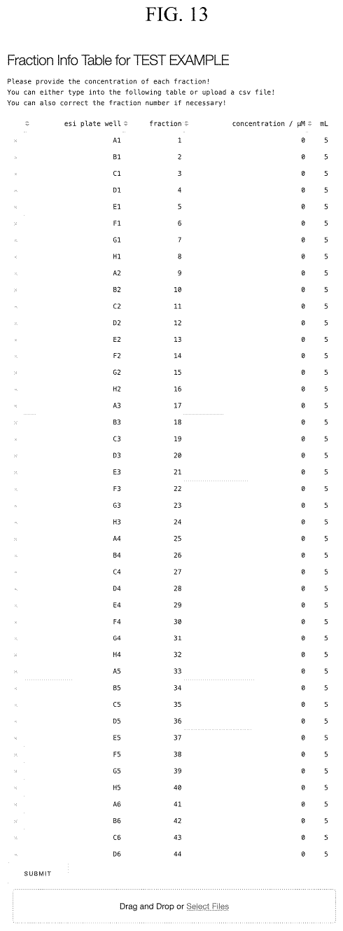

A web-based application was developed to manage and run Fraction Analyzer. A user can click on the “MS-based Fraction Analyzer” tab to choose using MS-based algorithms (illustrated in FIG. 18). The work order for fraction characterization can be typed into an input box and then submitted by clicking a SUBMIT button. The built-in algorithm described herein starts pulling both the raw MS data and the quantitative metric data for the processing run as described above and in FIG. 2. Depending on the Internet speed, this step might take a few minutes. An editable table (illustrated in FIG. 13) will show up with pre-populated fraction number, an empty column of concentration, and a pre-populated column of volume.

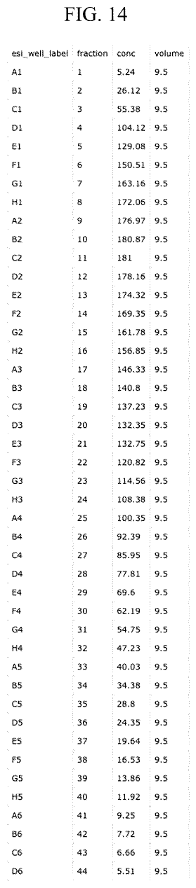

A user can type in the concentration and volume information, and also correct the fraction number if necessary. Moreover, by clicking the Select Files button or dragging a file into the upload region, users can also upload a csv file with concentration and volume information to avoid tedious typing or typo. An example of the concentration csv file is shown in FIG. 14. The csv file contains columns with the name conc and volume for the storage of concentration and volume values, respectively. The csv file also contains a column of either esi_well_label or fraction to be used as the index for fetching the information. When both esi_well_label and fraction columns exist, the former will be prioritized. Users have the opportunity to reload another file or manually correct any information in the Fraction Info Table, after the upload of the csv file.

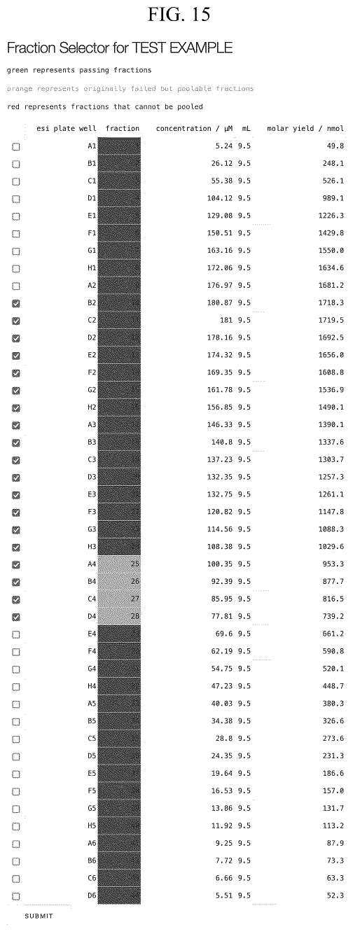

After SUBMIT button is clicked, another table will show up with checkbox in front of each fraction. The pre-selected fractions are the suggested pool decision that maximize pool yield and meet all the MS metric spec limits. The fractions are also color-coded (e.g., red, yellow or orange, and green), with legend above the table describing the quality of each fraction, as illustrated in FIG. 15.

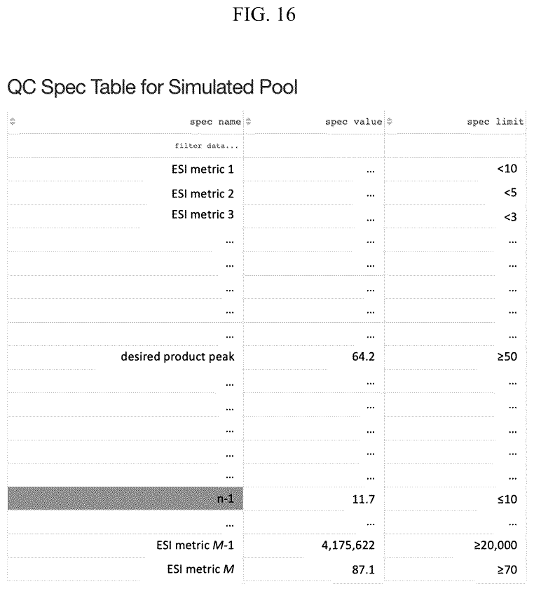

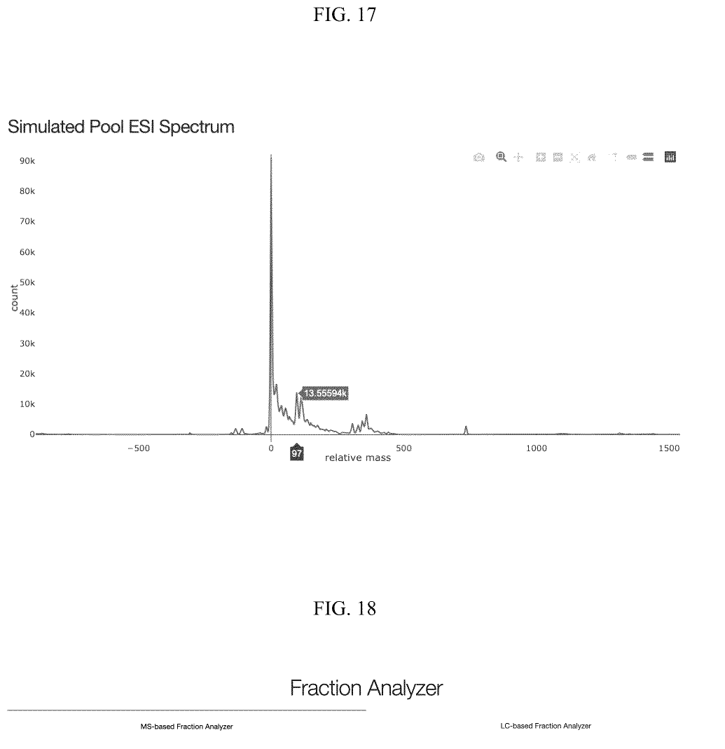

Based on user's selection in the Fraction Selector, the algorithm will update the simulated spec table (FIG. 16) and simulated spectrum (FIG. 17). In the spec table, any failing spec will be highlighted in red, as the n−1 in FIG. 16. The simulated spectrum as illustrated in FIG. 17 is also interactive, where user can zoom in and out. For example, a tooltip can provide information about relative mass and simulated counts.

After reviewing both the simulated spec table and simulate spectrum, users can decide on a suitable selection of pools to move on with manufacturing process.

User can make decisions based on preselected metric specification criteria as described herein. For example, the user can choose to combine certain fractions that will provide a certain yield with other quality-related features; or for example in most SPE fraction analysis, users prefer to select as much yield as possible to move on, even when some of the ESI specs are predicted to fail, because it is believed that downstream purification steps (e.g., HPLC) can help to clean up those impurities, but for HPLC fraction analysis users might want to sacrifice some yields for higher purity. Moreover, if LC-based fraction selection algorithms are used following the MS-based ones, users might select a subset of fractions that are expected to have good LC metrics based on their experiences in RNA manufacturing. Thus, the Fraction Analyzer methods disclosed herein provide an interactive method to help users make decisions during the manufacturing process.

In our example here, a user decided to further examine a subset of these fractions, namely, fractions #12 to #26 via the LC-based fraction selection algorithms. Such subset of fractions underwent LC analysis and the data were collected for each fraction.

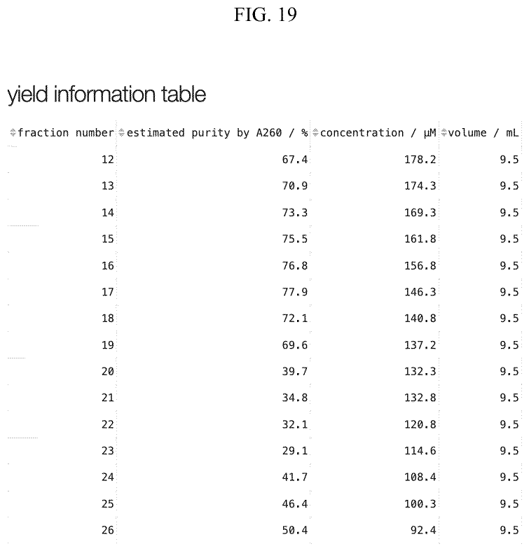

Once these data were available, a user can click on the “LC-based Fraction Analyzer” tab on the web-based application to use the LC-based algorithms (FIG. 18). Similar to the usage of MS-based algorithms, the work order for fraction characterization can be typed into an input box and then submitted by clicking the SUBMIT button. The built-in algorithm described herein starts pulling the LC chromatogram data for the processing run as described above in FIGS. 3A and 3B. Depending on the Internet speed, this step might take up to one minute. An editable table (illustrated in FIG. 19) will show up with pre-populated columns of fraction number, estimated purity, concentration, and volume. The estimated purity is calculated by the auto integrator described above in FIG. 6. The concentration and volume information are pulled from the fraction selection process performed with the MS-based algorithms, but can also be over-written by the user for any needed correction.

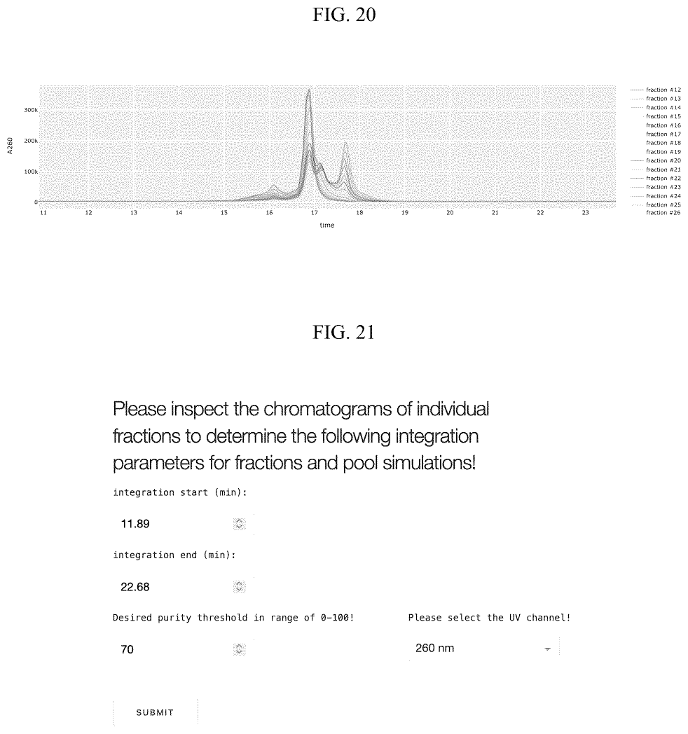

An overlay of LC chromatogram data for all the fractions involved in this LC-based selection process will also be shown (illustrated in FIG. 20). A user can examine this overlay plot to make a decision on some global integration parameters shown in FIG. 21. Such global integration parameters include integration start and end timestamps (i.e., t_(total start) and t_(total end), respectively, in Eq. 2), desired purity threshold (i.e., LC metric spec limit), and UV channel used for the analysis, if multiple channels are available from the LC data.

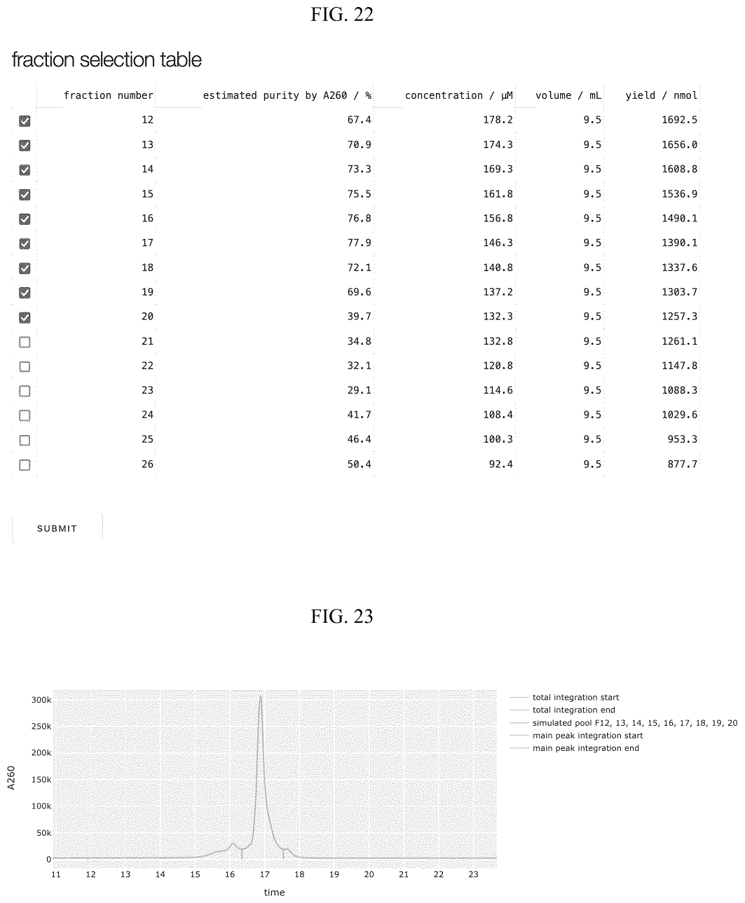

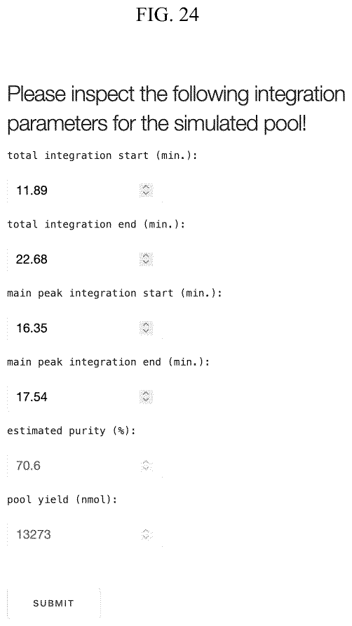

After the SUBMIT button is clicked, another table will show up with a checkbox in front of each fraction (FIG. 22). The pre-selected fractions are the suggested pool decisions that maximize pool yield and meet the LC metric spec limit. Simulated chromatogram data for the pool will also be shown to the user for examination (FIG. 23). The simulation is predicted by the pool chromatogram predictor algorithm as explained above in FIG. 5. A user can inspect the pool chromatogram and over-write some of the pool integration parameters (FIG. 24), if needed. More specifically, a user might want to modify the start and end timestamps for the integration of the main peak (i.e., t_(main start) and t_(main end), respectively, in Eq. 2). If any modification is made, the estimated pool purity and yield values in FIG. 24 will be updated once the SUBMIT button is clicked.

To help users navigate the balance between pool yield and LC metric, a summary plot of maximal poolable yields at different purity thresholds can also be provided (FIG. 25). At an interval of 5%, this plot examines all possible purity thresholds based on the purity values of individual fractions and depicts the yields of pools that meet such criteria and maximize the yield at the same time. Details such as the selected fractions and estimated purity value for a pool can be viewed in the hover information of each data point.

After reviewing both the simulated chromatogram and the estimated purity, users can decide on a suitable selection of pools to move on with manufacturing process. Normally, it is recommended for the user to physically combine aliquots from the selected subset of fractions to form a mock-pool. The mock-pool can be then used to provide characterization data (e.g., MS and/or LC data) to confirm the fraction selection decision satisfies quality criteria of the RNA sample.

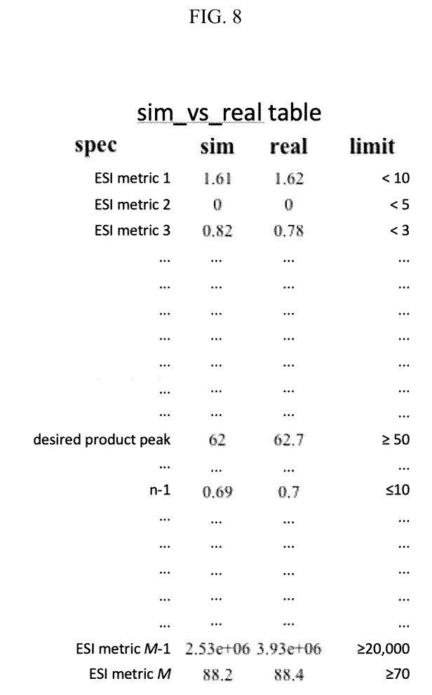

Shown in FIG. 8 is the comparison of spec values between the real pool sample (designated as real) and the pool sample simulated by Fraction Analyzer (designated as sim). Most of the spec names were redacted, but they were all species of chemical interest in oligonucleotide manufacturing, such as exemplified by the two unredacted rows, i.e., desired product peak and n-1. Similarly, spec limit values were also redacted. There was no significant difference in any of the specs.

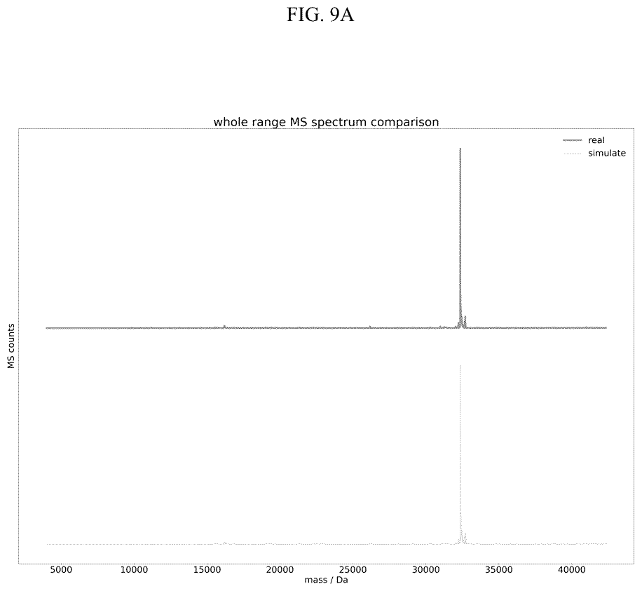

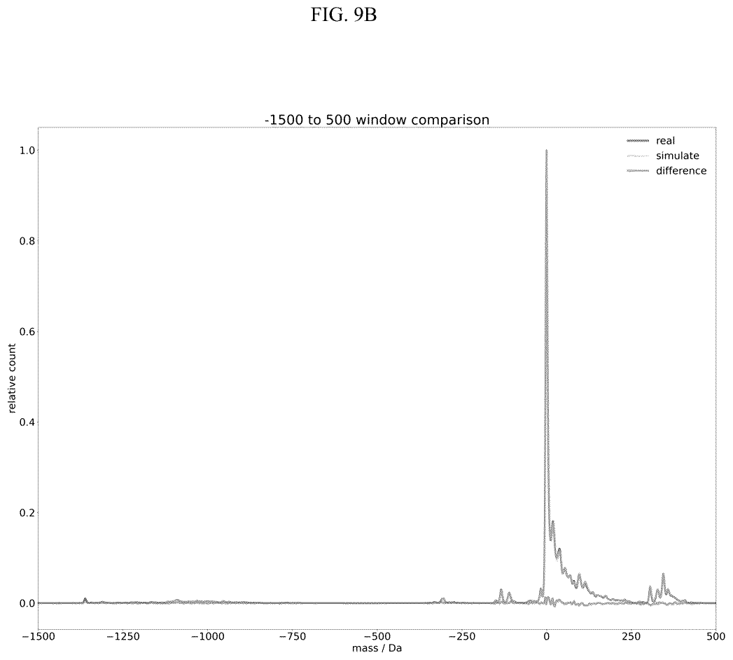

The real ESI spectrum was also compared with the one simulated by Fraction Analyzer. Shown in FIG. 9A is the full spectrum of real (top panel) and simulated (bottom panel) as well as a zoomed in version around expected molecular weight (FIG. 9B). Again, no significant difference was observed, which showed that Fraction Analyzer generated reliable simulation of our pool samples.

Guide RNA manufacturing starts with chemical synthesis where the RNA molecule is built one nucleotide at a time on a solid support. The synthesis starts with the attachment of the first nucleotide (usually 3′-terminal nucleotide) to the solid support. The remaining nucleotides are added step-by-step in the desired order using protected nucleotide building blocks. Each nucleotide is added to the growing RNA chain while still attached to the solid support. After the synthesis is complete, the RNA molecule, is cleaved from the solid support, typically using specific chemical treatment. Then the cleaved guide RNA undergoes deprotection to remove any chemical protecting groups used during synthesis. The deprotected RNA molecule is then purified using column-based purification methods, such as solid phase extraction (SPE) and high-performance liquid chromatography (HPLC), to remove impurities. In these purification steps, the individual portions of the RNA sample are collected as they elute from the chromatography column, which are known as fractions. During the purification process, the sample is separated into different components based on their physical and chemical properties, such as size, charge, hydrophobicity, or affinity. By analyzing fractions, the desired components of interest in the RNA sample can be isolated and concentrated, while unwanted impurities or interfering substances are separated. The purified RNA molecule is then aliquoted and stored at low temperature to maintain its stability until further use.

Depending on the manufacturing scale, it is common to have 10-50 different fractions involved in the fraction analysis process, where each fraction is characterized by 10-30 different metrics via mass spectrometry. For the mass spectrometry spectrum, the number of data points per fraction is 3,000 at the lower end and 40,000 at the upper end.

To estimate how long the disclosed procedures take manually, an individual researcher was timed to calculate one weighted average value for 10 fractions with the help of a calculator. It took 2 minutes to get the results, with most of the time spent on typing in the values.

Assuming that it takes 2 minutes to manually calculate a single data point (i.e., one metric, or the value for one mass in the spectrum), it will take 2,000 minutes (˜4 workdays, assuming one person works 8 hours per day) to simulate the metric values of all possible pools, and another 5.2 years (!) to simulate the spectrums of all possible pools, which is the simplest scenario with only 10 fractions, 10 metrics, and 3,000 data points per spectrum. At the other end of the possible scenarios (50 fractions, 30 metrics, 40,000 data points per spectrum), those two estimated times will be 1.3 years and 1,736 years.

With the disclosed procedures being performed by algorithms, all the simulation results are done within a couple of minutes.

Although inventive concepts have been described with reference to the embodiments illustrated in the attached drawing figures, equivalents may be employed and substitutions made herein without departing from the scope of the claims. Components illustrated and described herein are merely examples of a system/device and components that may be used to implement embodiments of the inventive concepts and may be replaced with other devices and components without departing from the scope of the claims. Furthermore, any dimensions, degrees, and/or numerical ranges provided herein are to be understood as non-limiting examples unless otherwise specified in the claims.

What is claimed is:

1. A method for manufacturing nucleic acid molecules, comprising:
obtaining, using a processor, information identifying a plurality of fractions from a nucleic acid synthesis procedure;
obtaining, using the processor, characterization information regarding each of the plurality of fractions,
the characterization information comprising liquid chromatography data for each of the plurality of fractions;
identifying, using the processor, a subset of the plurality of fractions to combine to generate a simulated pool based on a metric;
simulating, using the processor, a predicted metric for the simulated pool based on identifying the subset of the plurality of fractions to combine; and
providing, using the processor, information identifying the subset of fractions to a user to combine into a combined pool based on simulating the predicted metric for the simulated pool.

2. The method of claim 1, wherein the liquid chromatography data comprises chromatogram data for each of the plurality of fractions, and
wherein simulating a predicted metric for the simulated pool further comprises:
aligning the chromatogram data for each of the plurality of fractions, and
aggregating the aligned chromatogram data for each of the plurality of fractions based on aligning the chromatogram data to produce simulated pool chromatogram data.

3. The method of claim 2, wherein simulating a predicted metric for the simulated pool further comprises:
identifying a plurality of peaks in the simulated pool chromatogram data,
determining a main peak in the simulated pool chromatogram data based on identifying the plurality of peaks, and
determining the predicted metric for the simulated pool based on determining the main peak.

4. The method of claim 1, wherein obtaining characterization information further comprises:
obtaining characterization information comprising mass spectrometry data for each of the plurality of fractions.

5. The method of claim 4, wherein simulating a predicted metric for the simulated pool further comprises:
simulating the predicted metric for the simulated pool based on determining a weighted average of the mass spectrometry data for the subset of the plurality of fractions.

6. The method of claim 5, wherein determining a weighted average of the mass spectrometry data of the subset of the plurality of fractions further comprises:
weighting the average of the mass spectrometry data for the subset of the plurality of fractions based on a molarity of nucleic acids in each fraction of the subset of the plurality of fractions.

7. The method of claim 6, wherein simulating a predicted metric for the simulated pool further comprises:
generating a predicted mass spectrometry spectrum for the simulated pool.

8. The method of claim 6, wherein simulating a predicted metric for the simulated pool further comprises:
simulating a plurality of predicted metrics for the simulated pool, and
generating a table of the plurality of predicted metrics.

9. The method of claim 1, wherein identifying a subset of the plurality of fractions to combine to generate a simulated pool further comprises:
adding a fraction to the subset of fractions to generate a new subset of fractions based on determining the weighted average of the metric of the subset of the plurality of fractions, and
determining an updated metric for the new subset of the plurality of fractions.

10. The method of claim 1, wherein providing information identifying the subset of fractions to a user to combine into a combined pool further comprises:
receiving input from the user selecting a modified subset of fractions to combine into the combined pool,
wherein the modified subset of fractions is different from the subset of fractions.

11. The method of claim 1, wherein identifying a subset of the plurality of fractions to combine to generate a simulated pool based on a metric further comprises:
identifying a subset of the plurality of fractions to combine to generate a simulated pool based on identifying the simulated pool having a local optimum value.

12. The method of claim 1, further comprising:
combining the subset of fractions into the combined pool.

13. The method of claim 12, further comprising:
further processing the combined pool to generate a nucleic acid product.

14. The method of claim 1, wherein the nucleic acids comprise guide RNA (gRNA) molecules.

15. The method of claim 1, further comprising:
combining a portion of each of the subset of fractions into a mock combined pool,
obtaining further characterization information from the mock combined pool,
the further characterization information comprising at least one of liquid chromatography data or mass spectrometry data for the combined pool,
determining, based on the further characterization information, whether the mock combined pool satisfies a quality metric for the nucleic acid molecules.
